(19) United States
(12) Patent Application Publication
(10) Pub. No.: US 2025/0375177 A1
(43) Pub. Date: Dec. 11, 2025

(54) DYNAMIC IMAGING SYSTEM, DYNAMIC IMAGING METHOD, AND STORAGE MEDIUM

(71) Applicant: Konica Minolta, Inc., Tokyo (JP)

(72) Inventors: Keisuke KOEDA, Tokorozawa-shi (JP); Takeshi NUKANOBU, Tokyo (JP)

(21) Appl. No.: 19/234,894

(22) Filed: Jun. 11, 2025

(30) Foreign Application Priority Data
Jun. 11, 2024 (JP) 2024-094241

(51) Int. Cl. A61B 6/00

(52) U.S. Cl. CPC A61B 6/542; A61B 6/5205

(57) ABSTRACT

A dynamic imaging system includes a radiation source, a radiation detector, and a hardware processor. The dynamic imaging system obtains a still image consisting of a single frame and a dynamic image consisting of multiple frames by irradiating a subject with radiation emitted by the radiation source and detecting the radiation transmitted through the subject by the radiation detector. The hardware processor controls the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period. The hardware processor ends the one session of imaging, based on a total number of frames of the still image and the dynamic image.

### CROSS-REFERENCE TO RELATED APPLICATIONS

The entire disclosure of Japanese Patent Application No. 2024-094241 filed on June 11, 2024 is incorporated herein by reference in its entirety.

### TECHNICAL FIELD

The present invention relates to a dynamic imaging system, a dynamic imaging method, and a storage medium.

### BACKGROUND ART

There is known a radiographic imaging system intended for dynamic imaging of a subject outside an imaging room in a hospital (see Japanese Unexamined Patent Publication No. 2019-5073). The radiographic imaging system allows dynamic imaging of a patient who is difficult to move to an imaging room, such as a seriously injured patient, and is therefore usable.

There has been a demand of continuously capturing a long-time moving image in fluoroscopy, swallowing video fluorography, and so forth using a dynamic imaging system. However, the total dose in one time of dynamic imaging is limited from the viewpoint of risk management. Therefore, X-rays cannot be continuously emitted for a long time. To deal with this, there has been proposed to perform multiple times of dynamic imaging from the start to the end of single imaging, based on a specified imaging period and an imaging pause period.

In performing dynamic imaging, it is desirable to check the position of the patient before dynamic imaging to avoid re-imaging and to suppress an exposure dose. However, according to the related art, no consideration is given to capturing a still image for checking the position before dynamic imaging. Therefore, the total exposure dose that is the total of the exposure dose in dynamic imaging and the exposure dose in still image capturing cannot be managed.

To solve the above-described problem, an object of the present invention is to provide a dynamic imaging system, a dynamic imaging method, and a program that allow managing of the total exposure dose in an intermittent imaging mode that includes still image capturing and dynamic imaging.

### SUMMARY OF THE INVENTION

To achieve at least one of the abovementioned objects, according to an aspect of the present invention, there is provided a dynamic imaging system including: a radiation source; a radiation detector; and a hardware processor, wherein: the dynamic imaging system obtains a still image consisting of a single frame and a dynamic image consisting of multiple frames by irradiating a subject with radiation emitted by the radiation source and detecting the radiation transmitted through the subject by the radiation detector, the hardware processor controls the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period, and the hardware processor ends the one session of imaging, based on a total number of frames of the still image and the dynamic image.

According to an aspect of the present invention, there is provided a radiographic imaging method for a dynamic imaging system that includes a radiation source, a radiation detector, and a hardware processor and that obtains a still image consisting of a single frame and a dynamic image consisting of multiple frames by irradiating a subject with radiation emitted by the radiation source and detecting the radiation transmitted through the subject by the radiation detector, the method including: controlling the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period, and ending the one session of imaging, based on a total number of frames of the still image and the dynamic image.

According to an aspect of the present invention, there is provided a non-transitory computer-readable storage medium storing a program for a dynamic imaging system that includes a radiation source, a radiation detector, and a computer and that obtains a still image consisting of a single frame and a dynamic image consisting of multiple frames by irradiating a subject with radiation emitted by the radiation source and detecting the radiation transmitted through the subject by the radiation detector, wherein the program causes the computer to: control the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period; and end the one session of imaging, based on a total number of frames of the still image and the dynamic image.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 illustrates an example of a schematic configuration of a radiographic imaging system according to a first embodiment;

FIG. 2 illustrates a first irradiation pattern of X-rays emitted from a radiation source in the intermittent imaging mode according to the first embodiment;

FIG. 3 illustrates a second irradiation pattern of X-rays emitted from the radiation source in the intermittent imaging mode according to the first embodiment;

FIG. 4 illustrates a third irradiation pattern of X-rays emitted from the radiation source in the intermittent imaging mode according to the first embodiment;

FIG. 5 illustrates a fourth irradiation pattern of X-rays emitted from a radiation source in the intermittent imaging mode according to the first embodiment;

FIG. 6 is a flowchart as an example of operations of the radiographic imaging system in the intermittent imaging mode according to the first embodiment;

FIG. 7 illustrates an example of an examination screen displayed on a display part according to the first embodiment;

FIG. 8 illustrates an example of a warm-up operation of an imaging device and the radiation source in the intermittent imaging order according to the first embodiment;

FIG. 9 illustrates an example of operations of the radiation source and the imaging device when a still image switch is pressed in the intermittent imaging mode according to the first embodiment;

FIG. 10 illustrates an example of operations of the radiation source and the imaging device when a dynamic switch 24 is pressed in the intermittent imaging mode according to the first embodiment;

FIG. 11 illustrates an example of the examination screen that shows notification information and so forth according to the first embodiment;

FIG. 12 illustrates an example of a first stop operation of the radiation source and the imaging device at the end of the intermittent imaging mode according to the first embodiment;

FIG. 13 illustrates an example of a second stop operation of the radiation source and the imaging device at the end of the intermittent imaging mode according to the first embodiment;

FIG. 14 illustrates an example of a third stop operation of the radiation source and the imaging device at the end of the intermittent imaging mode according to the first embodiment; and

FIG. 15 is a flowchart as an example of operations of the radiographic imaging system in the intermittent imaging mode according to a second embodiment.

### DETAILED DESCRIPTION

A radiographic imaging system, a radiographic imaging method, and a program according to preferred embodiments of the present disclosure will be described in detail with reference to the accompanying drawings. However, the scope of the invention is not limited to the disclosed embodiments.

### First Embodiment

### Schematic Configuration Example of Radiographic Imaging System 100

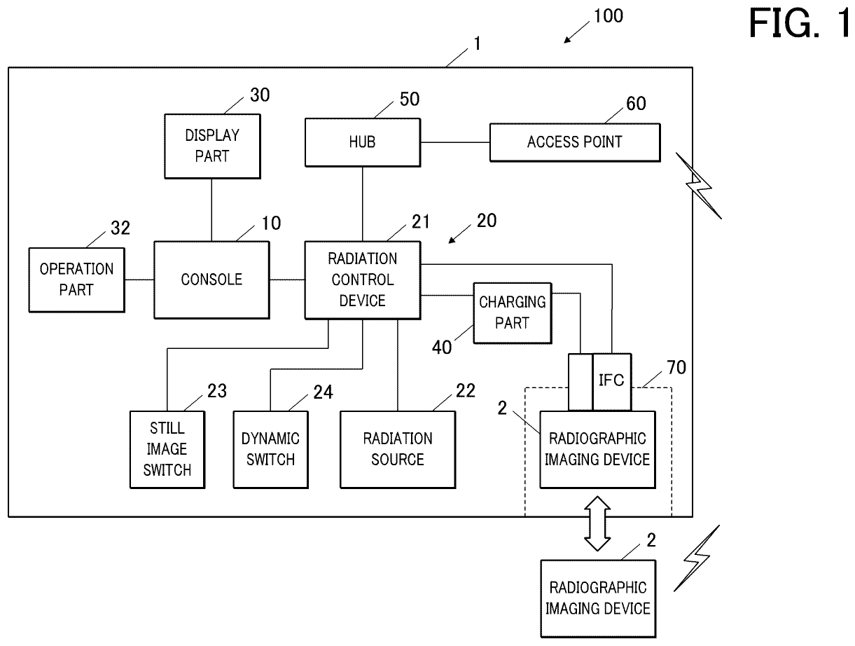

FIG. 1 illustrates an example of a schematic configuration of a radiographic imaging system 100 according to a first embodiment. As illustrated in FIG. 1, the radiographic imaging system 100 includes a medical cart 1 and a radiographic imaging device 2 (hereinafter referred to as an imaging device). The medical cart 1 includes a console 10, an irradiation device 20, a display part 30, an operation part 32, a charging part 40, a hub 50, an access point 60, and a housing 70.

The console 10 automatically sets imaging conditions, based on imaging order information transmitted from an RIS or the like. RIS is an abbreviation for Radiology Information System. A user, such as a doctor or a radiologist, may manually set the imaging conditions by manipulating the operation part. Examples of the imaging conditions include patient conditions regarding a subject, irradiation conditions regarding emission of radiation, and image reading conditions regarding reading of an image by the imaging device. The patient conditions include, for example, an imaging site, an imaging direction, and a physique. The irradiation conditions include, for example, a tube voltage (kV), a tube current (mA), an irradiation time (ms), a current-time product (mAs value), an irradiation field size (vertical and horizontal aperture size of a collimator), and a pulse frame rate. The image reading conditions include, for example, a pixel size, an image size, and a frame rate. The console 10 obtains image data of a radiographic image generated by the imaging device 2 and outputs the obtained image data to an external device such as a PACS via a network. PACS is an abbreviation for Picture Archiving and Communication System.

The irradiation device 20 includes a radiation control device 21 and a radiation source 22. The radiation control device 21 obtains the imaging conditions set by the console 10 or the like and transmits the obtained imaging conditions to the irradiation device 20 and the imaging device 2. The imaging conditions include, for example, the above-described irradiation conditions and image reading conditions. Based on the time in the radiation control device 21, the radiation control device 21 generates timing pulse signals that serve as a reference of emitting radiation from the radiation source 22 and sends the generated timing pulse signal to the radiation source 22. The radiation control device 21 performs time synchronization to adjust time with the imaging device 2.

The radiation control device 21 is connected to a still image switch 23 and a dynamic switch 24. The still image switch 23 is a hand switch, for example and is pressed by the user to start still image capturing. In response to being pressed by the user, the still image switch 23 generates an operation signal and outputs the generated operation signal to the radiation control device 21. The dynamic switch 24 is, for example, a foot switch and is pressed by the user to start dynamic imaging. In response to being pressed by the user, the dynamic switch 24 generates an operation signal and outputs the generated operation signal to the radiation control device 21. The configuration of the still image switch 23 and the dynamic switch 24 is not limited to the present example as long as the two systems of still image capturing and dynamic image capturing can be distinguished. Whether the still image switch 23 and the dynamic switch 24 are pressed (ON) or pulled (OFF) may be determined by the radiation control device 21 or by the console 10 based on an operation signal supplied by the radiation control device 21. Further, the still image switch 23 and the dynamic switch 24 may be connected to the console 10.

The radiation source 22 irradiates a subject with radiation (e.g., X-rays), based on the irradiation conditions, the timing pulse signals, and the like supplied from the radiation control device 21. The radiation source 22 generates radiation in a manner corresponding to the type of radiographic image (e.g., a still image or a dynamic image). Specifically, in generating a still image, the radiation source 22 emits radiation only once when the still image switch 23 is pressed once. In generating a dynamic image, the radiation source 22 performs dynamic imaging of repeatedly irradiating the subject with pulsed radiation at predetermined time intervals when the dynamic switch 24 is pressed once to obtain a series of images of the subject. Repetitive emission of pulsed radiation at predetermined time intervals is referred to as pulsed irradiation. The dynamic imaging includes obtaining a series of images of the subject by continuously irradiating the subject at a low dose rate in response to one imaging operation. Continuously applying radiation without interruption is referred to as continuous irradiation. A series of images obtained by dynamic imaging is called a dynamic image. Images constituting a dynamic image are called frames. The dynamic imaging includes moving image capturing but does not include capturing of a still image while displaying a moving image. Further, examples of a dynamic image includes a moving image but does not include still images captured while displaying a moving image.

The imaging device 2 detects the radiation emitted from the radiation source 22 of the irradiation device 20 and generates digital image data that shows the imaging region of the subject. The imaging device 2 is an example of a radiation detector. As the imaging device 2, for example, a portable FPD can be used. FPD is an abbreviation for Flat Panel Detector. The imaging device 2 includes an oscillator and communicates with the radiation control device 21 and performs time synchronization to adjust the time of the imaging device 2 to the time of the radiation control device 21. Time synchronization is performed when the imaging device 2 is housed in the housing 70. Using the time based on the time synchronization, the imaging device 2 generates a timing pulse signal that serves as a reference of imaging.

The synchronization between the irradiation device 20 and the imaging device 2 can be done by the following method. If the imaging device 2 is connected to the medical cart 1 via a wire, timing information generated by the imaging device 2 may be synchronized with timing information generated by the radiation control device 21. The imaging device 2 may maintain the state of synchronization with the radiation control device 21 even after the imaging device 2 is disconnected from the medical cart 1. That is, the radiation control device 21 and the imaging device 2 synchronize when they are connected to each other via a wire; whereas they continue to generate synchronization pulses to each other and run by themselves when they are connected wirelessly. The imaging device 2 may perform time synchronization communication with the radiation control device 21 over a local area network called IEEE 1588 to correct a time difference between the imaging device 2 and the radiation control device 21. The radiation control device 21 may perform synchronization between the radiation source 22 and the imaging device 2 by transmitting a synchronization signal to the radiation source 22 and the imaging device 2. The console 10 may perform synchronization between the radiation source 22 and the imaging system 2 by transmitting a synchronization signal to the radiation source 22 and the imaging device 2. If multiple synchronization methods are available, the methods may be switched from one method to a method with high synchronization accuracy.

The display part 30 is, for example, a display such as a liquid crystal display or an organic EL display. EL is an abbreviation for Electro Luminescence. Under the control of the console 10, the display part 30 displays a still image and a dynamic image captured by the imaging device 2 on an examination screen, which is described later. The display part 30 displays a GUI and so forth for receiving various input operations of the user. GUI is an abbreviation for Graphical User Interface. The display part 30 may be configured integrally with the housing of the medical cart 1, may be configured to be detachable from the housing, or may be installed in a place different from the medical cart 1.

The operation part 32 includes, for example, at least one of a mouse, a keyboard, a switch, and a button. The operation part 32 may be, for example, a touch screen integrally combined with a display or may be an interface that receives a voice input. The operation part 32 receives instructions corresponding to various types of input operations from the user, converts the received instructions into operation signals, and outputs the operation signals to the radiation control device 21.

The charging part 40 charges a built-in power source such as a battery provided in the imaging device 2, for example, when the imaging device 2 is stored in the housing 70. For example, the charging part 40 may be charged by electric power supplied from an external power source or by electric power supplied from a power source provided to the medical cart 1.

The hub 50 is, for example, a switching hub and has multiple ports. The hub 50 is connected to the radiation control device 21. The access point 60 is connected to the hub 50 via a communication cable. The access point 60 transmits and receives radio waves of a wireless LAN such as Wi-Fi (R). For example, when the imaging device 2 is detached from the medical cart 1 for use, the imaging device 2 is wirelessly connected to the access point 60 and sends and receives various kinds of data and signals to and from the medical cart 1.

The imaging device 2 is insertable to and removable from the housing 70. The housing 70 can house multiple imaging devices 2. When the imaging device 2 is housed in the housing 70, the imaging device 2 performs time synchronization with the radiation control device 21.

### Configuration and Functional Example of Console 10

Next, the configuration, functions, and so forth of the console 10 according to the first embodiment will be described in detail. The console 10 functions as a computer and includes a controller including a processor such as a CPU and a memory. CPU is an abbreviation for Central Processing Unit. The controller of the console 10 controls operations of components such as the irradiation device 20, the display part 30, and the operation part 32. The controller of the console 10 executes a program stored in the memory to perform processes such as a still image capturing mode, a dynamic imaging mode, and an intermittent imaging mode. In the present embodiment, the console mainly performs the processes including the intermittent imaging mode. The present invention is not limited thereto, though. For example, a control device other than the console 10, such as the radiation control device 21, may perform the processes including the intermittent imaging mode or may perform the processes including the intermittent imaging mode in cooperation with the console 10.

In the present embodiment, the intermittent imaging mode refers to an imaging mode in which still images and dynamic images are captured multiple times between the start to the end of one time of imaging, based on a specified imaging period and a specified imaging suspension period. The start of imaging is a timing at which both the imaging device 2 and the radiation source 22 become “ready” and X-ray irradiation is permitted in an intermittent imaging order. The end of imaging is a timing at which the intermittent imaging order ends; and still image capturing and dynamic imaging in this order are not available. The imaging period is a period from the start of imaging to the end of imaging. The console 10 performs the intermittent imaging mode by controlling the radiation source 22 and the imaging device 2. In the intermittent imaging mode, the console 10 controls the end of imaging, based on the total number of frames of still images and dynamic images.

The console 10 serves as a setting unit that sets imaging conditions to the radiation source 22. It is desirable that the console 10 does not accept a change in the imaging conditions to the radiation source 22 and the imaging device 2 between the start of imaging to the end of imaging in the intermittent imaging mode. Thus, the imaging conditions of the radiation source 22 and the imaging device 2 can be unified while the intermittent imaging mode is performed. In the intermittent dynamic mode, it is preferable that the console 10 captures still image and dynamic images under the same imaging conditions regarding irradiation conditions of one frame. The imaging conditions refer to a tube voltage (kV), a tube current (mA), a mAs value, an additional filter, and a scattered radiation removal grid. Since the still image capturing and the dynamic imaging are always performed under the same imaging conditions, the exposure dose can be easily managed. Further, the image quality per frame of still images and dynamic images can be equalized, so that a user can check the image quality of dynamic imaging beforehand using a still image.

### X-Ray Irradiation Pattern in Intermittent Imaging Mode

Next, an X-ray irradiation pattern in the intermittent imaging mode according to the first embodiment will be described.

FIG. 2 is a diagram illustrating a first irradiation pattern of X-rays emitted from the radiation source 22 in the intermittent imaging mode. In the first irradiation pattern, still image capturing is performed once, and dynamic imaging is performed once. For example, the frame rate is set to 15 fps, and the first specified number of frames is set to 300 frames. First, when the user presses the still image switch 23, still image capturing of the imaging region of the subject is performed. In the still image capturing, a still image constituted by one frame is obtained. The still image is displayed on the examination screen of the display part 30. The user checks the position of the patient while viewing the still image displayed on the examination screen. During this period, the intermittent imaging mode is suspended.

After confirming that the position of the patient is appropriate, the user proceeds to dynamic imaging. When the user presses the dynamic switch 24, dynamic imaging is started. During dynamic imaging, the dynamic switch 24 is pressed continuously. In dynamic imaging, a dynamic image consisting of 299 frames is obtained. In the present embodiment, when the total number of frames that is the total number of frames obtained in the still image capturing and in the dynamic imaging reaches the first specified number of frames, the dynamic imaging is ended. That is, the intermittent imaging mode ends. The intermittent imaging mode may end when the maximum imaging time (10 minutes) has elapsed even if the number of frames of the dynamic imaging does not reach the first specified number of frames.

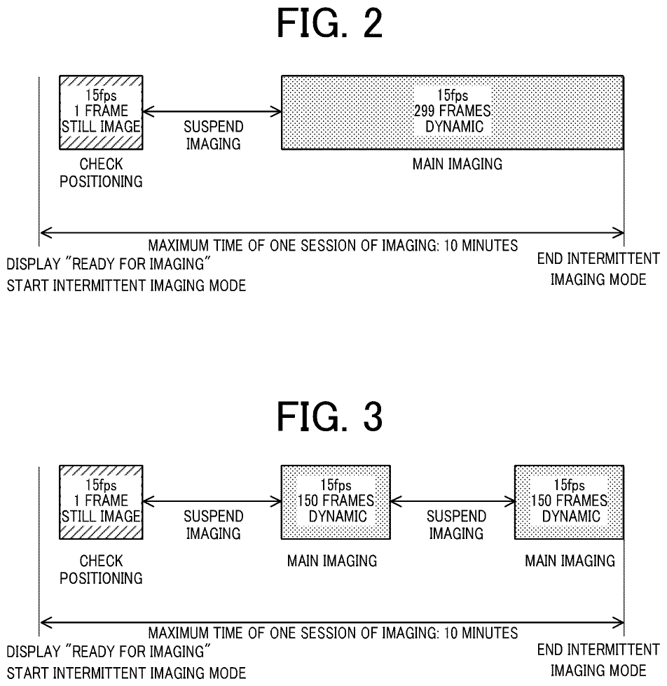

FIG. 3 is a diagram illustrating the second irradiation pattern of X-rays emitted from the radiation source 22 in the intermittent imaging mode. In the second irradiation pattern, still image capturing is performed once, and dynamic imaging is performed twice. For example, the frame rate is set to 15 fps, and the first specified number of frames is set to 300 frames. First, when the user presses the still image switch 23, still image capturing is performed. In the still image capturing, a still image constituted by one frame is obtained. The still image is displayed on the examination screen of the display part 30. The user checks the position of the patient while viewing the still image on the examination screen. During this period, the intermittent imaging is suspended.

After confirming that the position of the patient is appropriate, the user proceeds to dynamic imaging. When the user presses the dynamic switch 24, the first dynamic imaging is started. In the first dynamic imaging, a dynamic image consisting of 150 frames is obtained. The user turns off the dynamic switch 24 when a certain period has elapsed. The first dynamic imaging may automatically end at the timing when 150 frames are captured in the first dynamic imaging. During this period, the intermittent imaging mode is suspended. After a predetermined period, the second dynamic imaging is started in response to the user's pressing of the dynamic switch 24. In the second dynamic imaging, a dynamic image consisting of 149 frames is obtained. In the present embodiment, when the total number of frames that is the total number of frames obtained in one time of still image capturing and two times of dynamic imaging reaches the first specified number of frames, the dynamic imaging is ended. That is, the intermittent imaging mode ends.

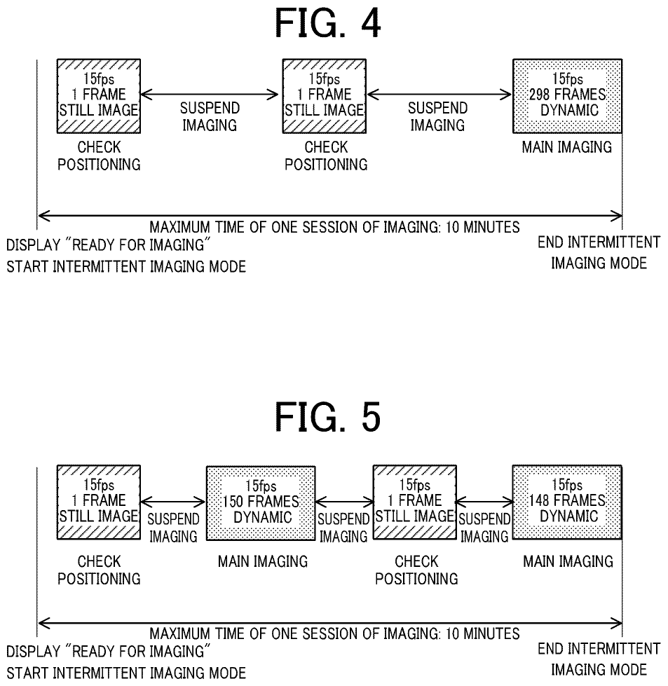

FIG. 4 is a diagram illustrating the third irradiation pattern of X-rays emitted from the radiation source 22 in the intermittent imaging mode. In the third irradiation pattern, the still image capturing is performed twice, and the dynamic imaging is performed once. For example, the frame rate is 15 fps, and the first specified number of frames is set to 300 frames. First, when the user presses the still image switch 23, first still image capturing is performed. In the still image capturing, a still image constituted by one frame is obtained. The still image is displayed on the examination screen of the display part 30.

The user checks the position of the patient while viewing the still image displayed on the examination screen. During this period, the intermittent imaging is suspended. If the position of the patient is not appropriate, the user guides the patient to the correct position and instructs the second still image capturing. When the user presses the still image switch 23, the second still image capturing is performed. In the still image capturing, a still image constituted by one frame is obtained. The still image is displayed on the examination screen of the display part 30. The user checks the position of the patient while viewing the still image on the examination screen. During this period, the intermittent imaging is suspended.

After confirming that the position of the patient is appropriate, the user proceeds to dynamic imaging. When the user presses the dynamic switch 24, dynamic imaging is started. In the dynamic imaging, a dynamic image consisting of 298 frames is obtained. In the present embodiment, when the total number of frames that is the total number of frames obtained in two times of still image capturing and one time of dynamic imaging reaches the first specified number of frames, the dynamic imaging is ended. That is, the intermittent imaging mode ends.

FIG. 5 illustrates a fourth irradiation pattern of X-rays emitted from the radiation source 22 in the intermittent imaging mode. In the fourth irradiation pattern, still image capturing is performed twice, and dynamic imaging is performed twice. For example, the frame rate is 15 fps, and the first specified number of frames is set to 300 frames. First, when the user presses the still image switch 23, the first still image capturing is performed. In the still image capturing, a still image constituted by one frame is obtained. The obtained still image is displayed on the examination screen of the display part 30. The user checks the position of the patient while viewing the still image on the examination screen. During this period, the intermittent imaging is suspended.

After confirming that the position of the patient is appropriate, the user proceeds to first dynamic imaging. When the user presses the dynamic switch 24, the first dynamic imaging is started. In the first dynamic imaging, a dynamic image consisting of 150 frames is obtained. The user turns off the dynamic switch 24 when a certain period of time has elapsed. The first dynamic imaging may automatically end at the timing when 150 frames are captured in the first dynamic imaging. During this period, the intermittent imaging mode is suspended.

If the position of the patient changes due to body motions such as a cough during the first dynamic imaging, the user needs to guide the patient to the correct position again and perform re-imaging. In such a case, the second still image capturing is performed. When the user presses the still image switch 23, the second still image capturing is performed. In the still image capturing, a still image constituted by one frame is obtained. The still image is displayed on the examination screen of the display part 30. The user checks the position of the patient while viewing the still image on the examination screen. During this period, the intermittent imaging is suspended.

After confirming that the position of the patient is appropriate, the user proceeds to second dynamic imaging. When the user presses the dynamic switch 24, second dynamic imaging is started. In the dynamic imaging, a dynamic image consisting of 148 frames is obtained. In the present embodiment, when the total number of frames that is the total number of frames obtained in two times of still image capturing and two times of dynamic imaging reaches the first specified number of frames, the dynamic imaging is ended. That is, the intermittent imaging mode ends.

### Operation Example of Radiographic Imaging System 100

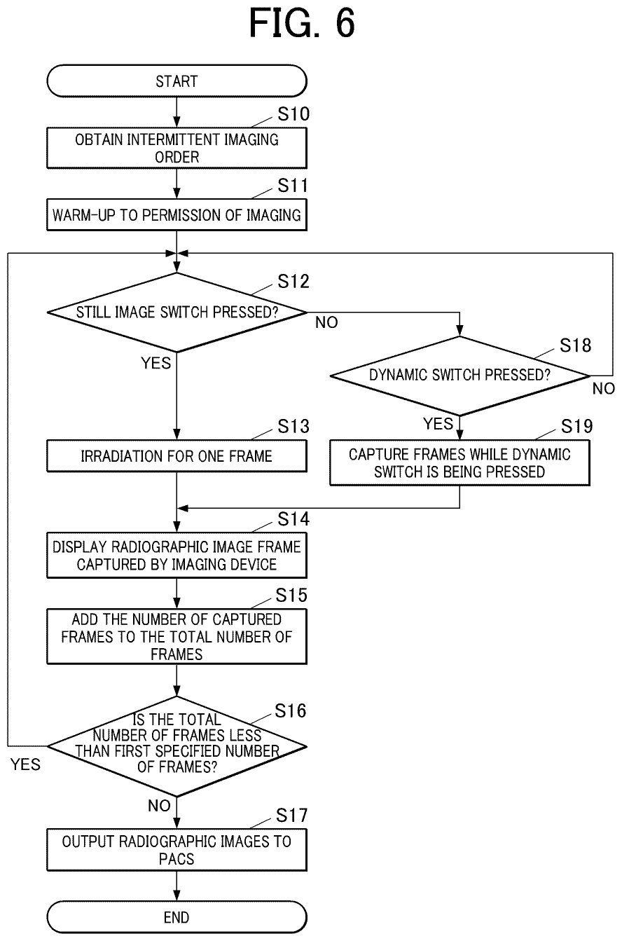

FIG. 6 is a flowchart as an example of operations of the radiographic imaging system 100 in the intermittent imaging mode according to the first embodiment. The console 10 that includes the controller performs the following process including a control step by executing a program stored in a not-illustrated storage section.

For example, when a radiology department receives examination information including an imaging order for a patient, the examination information is transmitted from the RIS to the medical cart 1. The display part 30 of the medical cart 1 displays a list screen (not illustrated) corresponding to the examination information. When a specific piece of examination information is selected by the user on the list screen, the display part 30 of the medical cart 1 displays an imaging screen corresponding to the examination information.

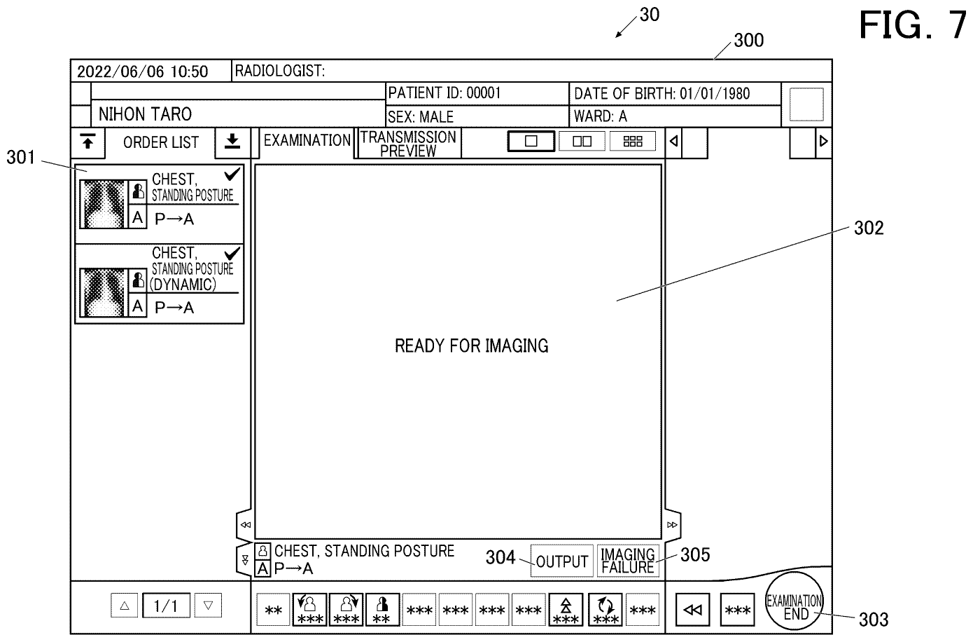

FIG. 7 illustrates an example of the examination screen 300 displayed on the display part 30 according to the first embodiment. The examination screen 300 includes an imaging order list 301, an image display section 302, an examination end button 303, an output button 304, an imaging failure button 305, and so forth. The imaging order list 301 is a section that shows a list of pieces of imaging order information included in the examination information selected on the list screen. The imaging order information includes an imaging region, an imaging direction, and an imaging category. The imaging categories include, for example, a still image capturing mode, a dynamic imaging mode, and an intermittent imaging mode that is a combination of still image capturing and dynamic imaging. The imaging categories further include a low-exposure dynamic imaging mode and a low-exposure intermittent imaging mode. Herein, when the dynamic imaging mode and the intermittent imaging mode are categorized as a group A and the low-exposure dynamic imaging mode and the low-exposure intermittent imaging mode are categorized as a group B, the maximum number of capturable frames may be set as B>A.

The image display section 302 is a section that shows a radiographic image captured by the imaging device 2. The image display section 302 shows information indicating the status of the system before imaging, such as “waiting for permission of exposure” and “ready for imaging”, for example. Although FIG. 7 shows “ready for imaging” for the sake of convenience, no words are displayed at this stage. The examination end button 303 is a button for ending the examination. The output button 304 is a button for outputting radiographic images including still images and dynamic images captured by the imaging device 2 to an external device such as a PACS 40. The imaging failure button 305 is a button for discarding a radiographic image captured by the imaging device 2 without outputting the radiographic image.

Referring back to FIG. 6, the user selects a specific imaging order from the imaging order list 301 on the examination screen 300 by operating the operation part 32. For example, when the user selects imaging order information indicating the intermittent imaging, the console 10 obtains intermittent imaging order information as the imaging order (step S10).

When obtaining the intermittent imaging order information, the console 10 instructs the imaging device 2, the radiation source 22, and so forth to warm up (step S11). When warm-up of the imaging device 2, the radiation source 22, and so forth is completed, the console 10 displays a message “ready for imaging” in the image display section 302 of the examination screen 300, as illustrated in FIG. 7. Further, the console 10 sets irradiation conditions to the radiation source 22, based on the selected intermittent imaging order, and sends image reading conditions to the imaging device 2 via the access point 60 or the like.

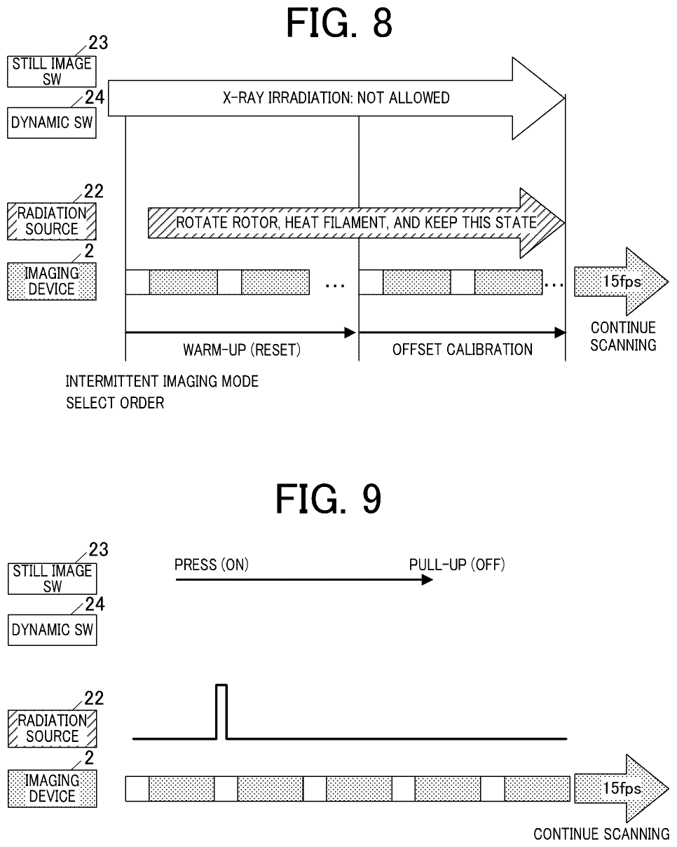

FIG. 8 illustrates an example of warm-up operations of the imaging device 2 and the radiation source 22 in the intermittent imaging order according to the first embodiment. When the radiation source 22 receives the instruction of warm-up from the radiation control device 21, the radiation source 22 drives the rotor, heats the filament, and maintains this state. When the imaging device 2 receives the instruction of warm-up from the console 10, the imaging device 2 performs warm-up (reset) and offset calibration. During the warm-up operations of the imaging device 2 and the radiation source 22, X-ray irradiation is not permitted even if the user presses the still image switch 23 or the dynamic switch 24. When the warm-up operations are completed, the imaging device 2 proceeds to an automatic detection mode. In the automatic detection mode, an accumulation operation and a readout operation are performed according to a set frame rate.

Referring to FIG. 6, the console 10 determines whether the still image switch 23 has been pressed by the user (step S12). When determining that the still image switch 23 has been pressed by the user, the console 10 proceeds to step S13. In this case, the console 10 controls the radiation control device 21 to emit X-rays corresponding to one frame toward the subject from the radiation source 22 (step S13).

FIG. 9 illustrates an example of operations of the radiation source 22 and the imaging device 2 when the still image switch 23 is pressed in the intermittent imaging mode according to the first embodiment. When the still image switch 23 is pressed, the radiation source 22 emits one pulse of X-rays corresponding to one frame, regardless of the pressing time of the still image switch 23. The imaging device 2 repeatedly performs the accumulation operation and the reading operation at the frame rate of 15 fps, for example. The imaging device 2 detects one pulse of X-rays emitted by the radiation source 22 at the timing of the reading operation and obtains one frame of a radiographic image. The console 10 proceeds to step S14 after obtaining the still image.

When determining in step S12 that the still image switch 23 has not been pressed by the user, the console 10 proceeds to step S18. The console 10 determines whether the dynamic switch 24 has been pressed (step S18). When determining that the dynamic switch 24 has been pressed, the console 10 proceeds to step S19. In this case, the console 10 controls the radiation control device 21 to emit pulsed X-rays toward the subject from the radiation source 22 (step S19).

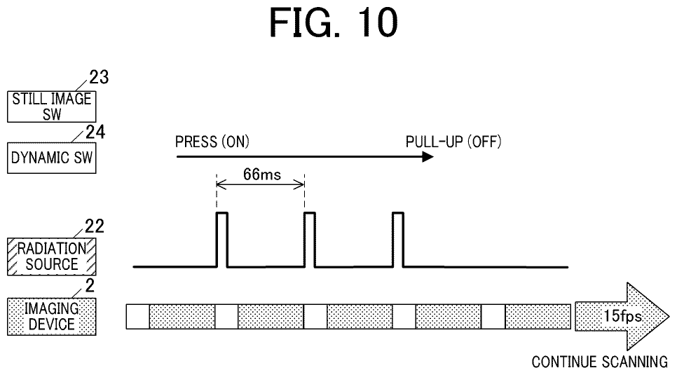

FIG. 10 illustrates an example of operations of the radiation source 22 and the imaging device 2 when the dynamic switch 24 is pressed in the intermittent imaging mode according to the first embodiment. When the dynamic switch 24 is pressed, the radiation source 22 continuously emits pulsed X-rays at 15 fps while the dynamic switch 24 is pressed, for example. The imaging device 2 repeatedly performs the accumulation operation and the reading operation at the same frame rate of 15 fps as the radiation source 22. The imaging device 2 detects X-rays emitted from the radiation source 22 in the reading operation at 15 fps to obtain a dynamic image consisting of multiple frames.

In the dynamic imaging, the frame rate of the imaging device 2 may be set to 15 fps, and the frame rate of the radiation source 22 may be set to 7.5 fps. The frame rate of 7.5 fps is half the frame rate of the imaging device 2. The radiation source 22 can emit X-rays while skipping the reading operation of the imaging device 2. Thus, the dose of X-rays emitted by the radiation source 22 can be reduced, so that the exposure dose can be reduced. Further, since the amount of X-rays to be detected by the imaging device 2 is reduced, the size of image data can be reduced, and the time for capturing a dynamic image can be extended. Further, since the frame rate of the imaging device 2 is unchanged, offset calibration for each changing is not required. Thus, imaging modes can be swiftly switched by simply switching X-ray irradiation between 15 fps and 7.5 fps.

Returning to FIG. 6, the console 10 displays the frame captured by the imaging device 2 in the image display section 302 of the examination screen 300 of the display part 30 (step S14). The image data of the frame captured by the imaging device 2 is, for example, wirelessly transmitted and received by the access point 60 of the medical cart 1. When a still image is captured in the intermittent imaging mode, a still image is displayed on the display part 30, for example. The user checks the position of the patient while viewing the still image. When a dynamic image is captured in the intermittent imaging mode, a dynamic image is displayed on the display part 30. The user makes diagnosis regarding the imaging region by viewing the dynamic image.

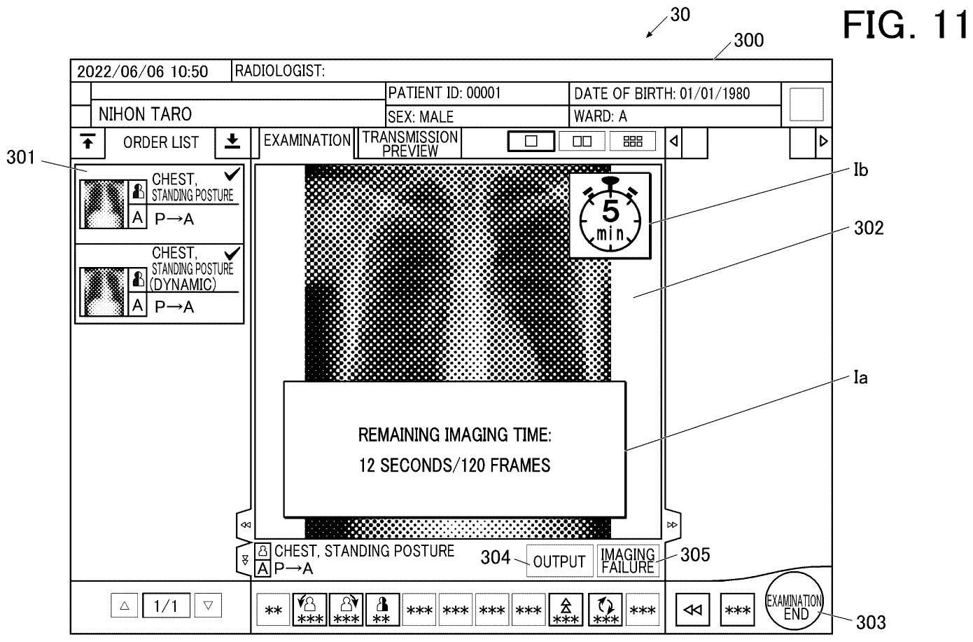

Herein, the console 10 may display, on the display part 30, notification information that notifies the user of the imaging state of the intermittent imaging mode. FIG. 11 illustrates an example of the examination screen 300 that shows first notification information Ia and so forth according to the first embodiment. The display part 30 displays the first notification information Ia that notifies the user of the imaging state and so forth in the intermittent imaging mode on the lower part of the image display section 302 of the examination screen 300. The display part 30 is an example of a notification unit. Examples of the first notification information Ia include the number of remaining frames to be obtained and the remaining imaging time in the intermittent imaging mode. Specifically, as the first notification information Ia, the text “Remaining imaging time: 12 seconds/120 frames” is displayed in the image display section 20) 302. The user can accurately grasp the remaining imaging time and so forth by checking the first notification information Ia on the examination screen 300 in performing the intermittent imaging mode.

Herein, the number of remaining frames is obtained by the following Expression (1).

The number of remaining frames=The current total number of frames−The first specified number of frames determined beforehand (1)

The remaining imaging time is obtained by the following expression (2).

The remaining imaging time=The remaining number of frames×(1×frame rate) (2)

Further, the display part 30 displays second notification information Ib that notifies the user of the imaging state and so forth in the intermittent imaging mode on the upper part of the image display section 302 of the examination screen 300. The second notification information Ib includes, for example, an elapsed time from the start of imaging to the present in the intermittent imaging mode. Specifically, as the second notification information Ib, the words “5 min” indicating the time elapsed from the start of imaging to the present in the intermittent imaging mode is displayed in the image display section 302. The second notification information Ib may indicate, for example, a remaining imaging time other than the elapsed time. The first notification information Ia and the second notification information Ib may be information other than characters, numbers, notes, and figures displayed on the display part 30. For example, the first notification information Ia and the second notification information Ib may be sound or vibration. In this case, the console 10 outputs a voice, a buzzer sound, vibration, or the like when the remaining imaging time reaches a certain value.

Referring to FIG. 6, the console 10 adds the number of frames corresponding to the number of pulses emitted by the radiation sources 22 to the total number of frames (step S15). Specifically, when one frame is captured by a press of the still image switch 23, the console 10 adds one frame to the total number of frames. Similarly, when multiple frames are captured by a press of the dynamic switch 24, the console 10 adds the number of the captured frames to the total number of frames. The console 10 may obtain information relating to frames from the imaging device 2 or may obtain information relating to the number of emitted pulses from the irradiation device 20.

The console 10 determines whether the added total number of frames is less than the first specified number of frames (upper limit value) that is set beforehand (step S16). That is, the console 10 determines whether the total number of frames that is the total of the number of frames captured in still image capturing and the number of frames captured in dynamic imaging is less than the first specified number of frames. The first specified number of frames is determined based on the total dose in one time of imaging from the viewpoint of risk management. The first specified number of frames can be determined depending on imaging conditions, such as a frame rate. The first specified number of frames may be set for each imaging order. The first specified number of frames may be set before shipment or may be set after shipment by a user or the like using the operation part 32 or the like. A specified value for determining whether the exposure dose in the intermittent imaging mode is within the allowable range may be a threshold value other than the first specified number of frames. For example, the specified value may be a specified imaging time calculated based on the first specified number of frames and the frame rate. Thus, imaging can be managed so that the total actual imaging time in the intermittent imaging mode does not exceed the predetermined imaging time.

When determining that the total number of frames is less than the first specified number of frames set in advance, the console 10 returns to step S12. This is a case where the number of captured frames has not reached the upper limit number of frames in the intermittent imaging mode. As described above, the console 10 repeats still image capturing and dynamic imaging in the intermittent imaging mode until the total number of frames reaches the first specified number of frames.

On the other hand, when determining that the total number of frames is not less than the preset first specified number of frames, the console 10 proceeds to step S17. This is a case where the number of captured frames has reached the upper limit value in the intermittent imaging mode. In this case, the radiation source 22 and the imaging device 2 stop imaging.

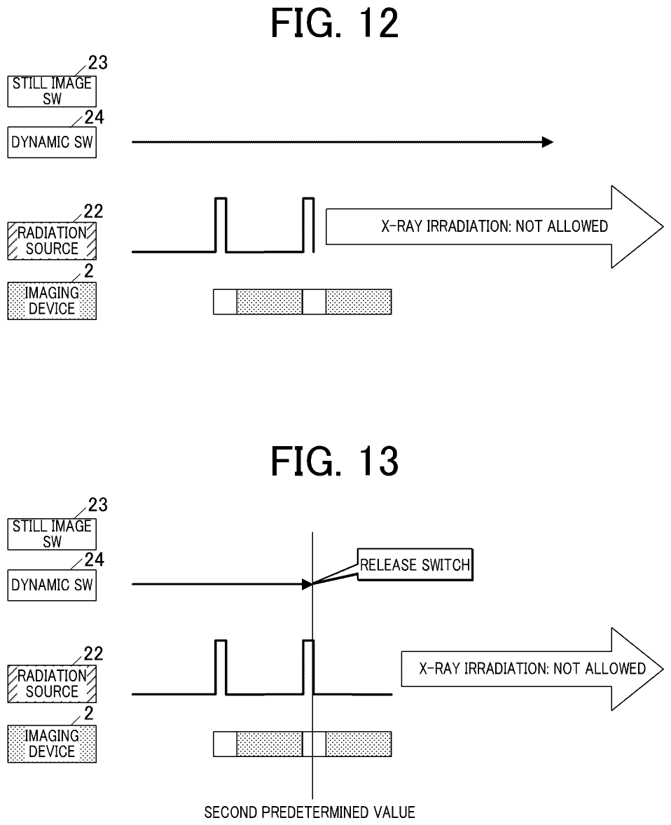

FIG. 12 illustrates an example of a first stop operation of the radiation source 22 and the imaging device 2 at the end of the intermittent imaging mode according to the first embodiment. When the total number of frames reaches the first specified number of frames during dynamic imaging in the intermittent imaging mode, the radiation source 22 stops X-ray irradiation in accordance with an instruction from the console 10. After the total number of frames reaches the first specified number of frames, X-ray emission is not permitted. Therefore, even when the dynamic switch 24 is pressed by the user, X-rays are not emitted. The imaging device 2 performs the readout operation of the last X-rays emitted from the radiation source 22 in accordance with instructions from the radiation control device 21. Thereafter, the imaging device 2 stops the readout operation. The imaging device 2 may continue the readout operation after reading out the last X-rays emitted by the radiation source 22 depending on the setting of the readout operation and the accumulation operation.

FIG. 13 illustrates an example of a second stop operation of the radiation source 22 and the imaging device 2 at the end of the intermittent imaging mode according to the first embodiment. In the following case of the second stop operation, dynamic imaging is stopped before the total number of frames captured in the intermittent imaging mode reaches the first specified number of frames. In the present embodiment, a second specified number of frames is defined as a specified value of the number of frames that is before the total number of frames reaches the first specified number of frames and that is smaller than the first specified number of frames. For example, assume a case where dynamic imaging is stopped immediately before the number of captured frames reaches the first specified number of frames. In such a case, there are a small number of remaining frames to be captured, and resuming dynamic imaging may result in unnecessary exposure. Therefore, when the number of frames after the resumption of dynamic imaging is equal to or greater than the second specified number of frames, the console 10 stops the dynamic imaging. The console 10 serves as a changing unit and can change the second predetermined number of frames for each session of imaging (one time of imaging start to one time of imaging end).

In dynamic imaging in the intermittent imaging mode, when the dynamic switch 24 is turned off before the total number of frames reaches the first specified number of frames, the console 10 determines whether or not the total number of frames is equal to or greater than the second specified number of frames. When the total number of frames is equal to or greater than the second specified number of frames, the console 10 stops the dynamic imaging. For example, this is a case where the first specified number of frames is 300 frames, the second specified number of frames is 290 frames, and the total number of frames is 292 frames. In this case, the radiation source 22 stops irradiation of X-rays in accordance with an instruction from the console 10. Thus, after the total number of frames reaches the second specified number of frames, X-ray irradiation is not permitted. Therefore, even when the dynamic switch 24 is pressed by the user, X-rays are not emitted. The imaging device 2 performs the readout operation of the last X-rays emitted from the radiation source 22 in accordance with instructions from the radiation control device 21. Thereafter, the imaging device 2 stops the readout operation. The imaging device 2 may continue the readout operation after reading out the last X-rays emitted by the radiation source 22 depending on the setting of the readout operation and the accumulation operation.

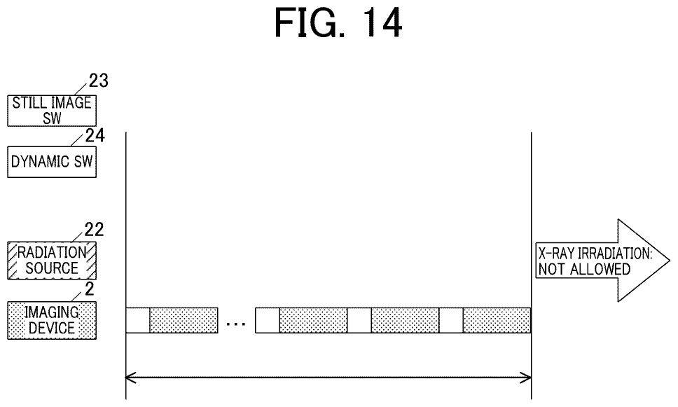

FIG. 14 illustrates an example of a third stop operation of the radiation source 22 and the imaging device 2 at the end of the intermittent imaging mode according to the first embodiment. For example, after the intermittent imaging mode is started, X-rays may not be emitted by the radiation source 22 depending on the condition of the patient. When the intermittent imaging mode is started, the imaging device 2 repeatedly performs the accumulation operation and the reading operation according to the set frame rate. If the imaging time from the start of the intermittent imaging mode exceeds a preset specified time, the console 10 forcibly stops the intermittent imaging mode. The specified time can be set as desired in consideration of the examination time of other patients, for example. Further, to prevent an image quality abnormality caused by offset correction, a value that determines an acquisition interval between an offset calibration image and an image to be captured may be set. When the imaging time exceeds the specified time, the imaging device 2 stops the accumulation operation and the readout operation corresponding to the frame rate in accordance with instructions by the console 10. After the specified time has elapsed, X-rays are not emitted even if the dynamic switch 24 is pressed by the user.

Referring to FIG. 6, the console 10 outputs the dynamic image obtained in the intermittent imaging mode to an external device, such as a PACS (step S17). In this case, the console 10 outputs, to the PACS or the like, image data to which supplementary information including patient information and inspection information has been added. Further, the console 10 may store the image data to which the supplementary information has been added in a not-illustrated memory in the medical cart 1. The image data may further include a still image captured for checking the positioning in the intermittent imaging mode. In a case where dynamic images are captured multiple times, the console 10 may connect the dynamic images of the respective times to generate one series of dynamic images.

In a case where the intermittent imaging mode including still image capturing and dynamic imaging is performed in the first embodiment, the total number of frames is calculated that is the total of the number of frames obtained by the still image capturing and the number of frames obtained by the dynamic imaging. Since the console 10 ends the intermittent imaging mode when the total number of frames reaches the first specified number of frames set beforehand, the console 10 can control emission of X-rays such that the total number of frames does not exceed the first specified number of frames. Thus, according to the first embodiment, the total exposure dose of still imaging and dynamic imaging can be taken into consideration, and the exposure dose in one time of imaging (examination) can be accurately managed.

### Second Embodiment

In the second embodiment, the still image switch 23 and the dynamic switch 24 are not separately provided as in the first embodiment. Both the still image capturing and the dynamic imaging are performed by pressing one exposure switch. In the following description, components substantially common to those of the first embodiment are denoted by the same reference numerals, and common description will be omitted or simplified.

### Operation Example of Radiographic Imaging System 100

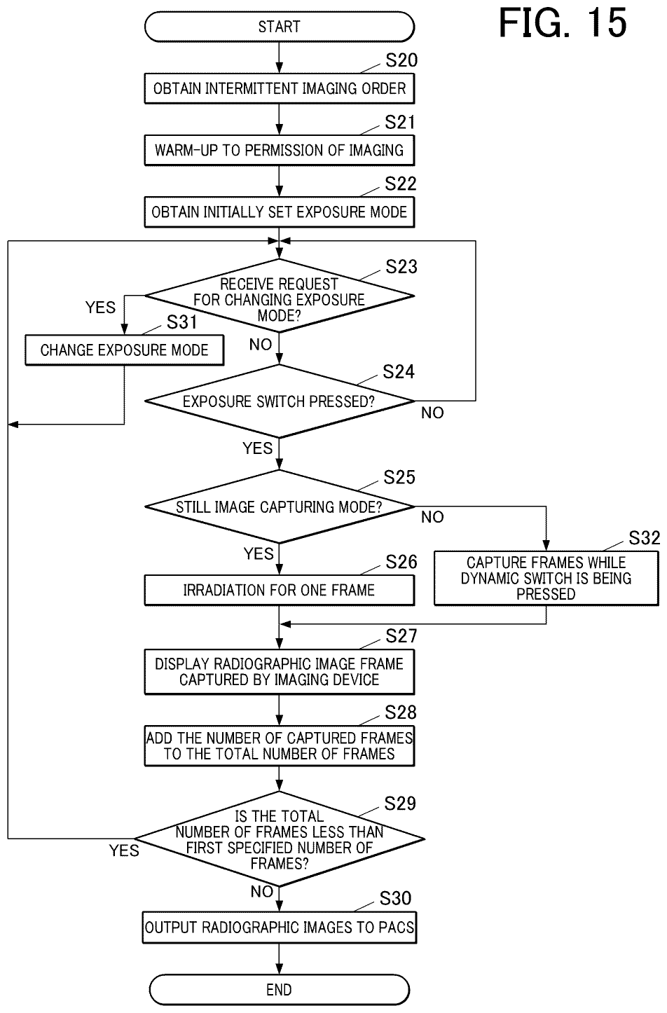

FIG. 15 is a flowchart as an example of operations of the radiographic imaging system 100 in the intermittent imaging mode according to the second embodiment. The console 10 that includes the controller performs the following process including a control step by executing a program stored in a not-illustrated storage section.

When an intermittent imaging order is selected on the examination screen 300 by the user, the console 10 obtains intermittent imaging order information as an imaging order (step S20).

When obtaining the intermittent imaging order information, the console 10 instructs the imaging device 2, the radiation source 22, and so forth to warm up (step S21). Further, the console 10 sets irradiation conditions to the radiation source 22, based on the selected intermittent imaging order information, and sends image reading conditions to the imaging device 2 via the access point 60 or the like.

The console 10 obtains the initially set exposure mode for the selected intermittent imaging order (step S22). For example, when the type of imaging order is an intermittent imaging order, the exposure mode includes still image capturing and dynamic imaging. The initially set exposure mode in the intermittent imaging order includes still image capturing because the position of a patient is checked first.

The console 10 determines whether there is a request to change the exposure mode (step S23). When determining that no request is made to change the exposure mode, the console 10 proceeds to step S24. For example, when imaging in the intermittent imaging mode is started, still image capturing that is initially set in the exposure mode is performed. In this case, the console 10 proceeds to step S24 without changing the exposure mode.

When determining that there is a request to change the exposure mode, the console 10 proceeds to step S31. For example, in the intermittent imaging mode, dynamic imaging is subsequently performed after still image capturing ends. In this case, the console 10 receives, from the radiation control device 21 or the like, a request for changing the exposure mode from still image capturing to moving image capturing.

The console 10 changes the exposure mode (step S31). For example, when still image capturing ends in the intermittent imaging mode, the console 10 changes the exposure mode from still image capturing to dynamic imaging.

The console 10 determines whether the exposure switch 25 has been pressed by the user (step S24). An operation signal corresponding to pressing of the exposure switch 25 by the user is supplied to the console 10 via, for example, the radiation control device 21. If the console 10 determines that the exposure switch 25 has been pressed by the user, the console 10 proceeds to step S25.

The console 10 determines whether the current exposure mode is still image capturing (step S25). When determining that the current exposure mode is still image capturing, the console 10 proceeds to step S26. The console 10 controls the radiation control device 21 to cause the radiation source 22 to emit X-rays corresponding to one frame toward the subject, based on the imaging instruction regarding the still image capturing (step S26). After completing step S26, the console 10 proceeds to step S27.

When determining in step S25 that the exposure mode is not still image capturing, the console 10 proceeds to step S32. That is, this is a case where the exposure mode is set to dynamic imaging. The console 10 controls the radiation source controller 21 to cause the radiation source 22 to continuously emit pulsed X-rays toward the subject while the exposure switch is being pressed, based on the imaging instruction related to dynamic imaging (step S32). After completing step S26, the console 10 proceeds to step S27.

The console 10 displays the frame captured by the imaging device 2 on the image display section 302 of the examination screen 300 of the display part 30 (step S27). Specifically, the image data of the frame captured by the imaging device 2 is wirelessly transmitted and received by the access point 60 or the like of the medical cart 1. For example, when the exposure mode is still image capturing, a still image is displayed on the display part 30. The user checks the position of the patient while viewing the still image. In a case where the exposure mode is dynamic imaging, a dynamic image is displayed on the display part 30. The user makes diagnosis regarding the imaging region by viewing the dynamic image.

The console 10 adds the number of frames corresponding to the number of pulses emitted by the radiation sources 22 to the total number of frames (step S28). Specifically, when one frame is captured by a press of the still image switch 23, the console 10 adds one frame to the total number of frames. In a case where multiple frames are captured by the press of the dynamic switch 24, the console 10 adds the multiple frames to the total number of frames.

The console 10 determines whether the added total number of frames is less than the first specified number of frames (upper limit value) that is set beforehand (step S29).

When determining that the total number of frames is less than the first specified number of frames set beforehand, the console 10 returns to step S23. Specifically, this is a case where the number of captured frames has not reached the upper limit value in the intermittent imaging mode. In this case, as described above, the console 10 repeats still image capturing and dynamic imaging in the intermittent imaging mode until the total number of frames reaches the first specified number of frames.

On the other hand, when determining that the total number of frames is not less than the preset first specified number of frames, the console 10 proceeds to step S30. This is a case where the number of captured frames has reached the upper limit value in the intermittent imaging mode. In this case, the radiation source 22 and the imaging device 2 stop imaging.

The console 10 outputs the image data corresponding to the dynamic image obtained in the intermittent imaging mode to the external device such as the PACS (step S30). Herein, the console 10 outputs, to the PACS or the like, image data to which supplementary information including patient information and inspection information has been added. The image data may further include a still image captured for checking the positioning in the intermittent imaging mode. In a case where dynamic images are captured multiple times, the console 10 may connect the dynamic images of the respective times to generate one series of dynamic images.

The second embodiment can have similar operational effects to the first embodiment described above. That is, since the total exposure dose of still image capturing and dynamic imaging can be considered in the intermittent imaging mode, the exposure dose in one time of imaging (examination) can be accurately managed. Further, since one exposure switch is used to generate a trigger for starting still image capturing and dynamic imaging, the configuration of the radiographic imaging system 100 can be simplified. In addition, since only one exposure switch is provided, erroneous exposure due to an operation error can be avoided, and operations can be simplified.

The following process can also be added to the flowcharts in FIG. 6 and FIG. 15, which illustrate flows in the intermittent imaging mode.

Before displaying a frame on the display part 30, it is preferable that the console 10 performs at least one of offset correction, gain correction, defect correction, afterimage correction, and gradation correction. When performing offset correction, the console 10 may obtain an image for offset correction (dark image) at the time of the first intermittent imaging and perform correction with the same offset correction image during imaging. Thus, in offset correction, internal processing can be simplified and accelerated. The console 10 may obtain an offset correction image for each group of still images and dynamic images in the intermittent imaging mode.

Frame information may be overlaid on an image. The frame information indicates which session of dynamic imaging the frame belongs to, or the frame information or indicates what the frame number is and to which session of dynamic imaging the frame belongs to. Instead of being overlaid on the image, the frame information may be added to supplementary information of the image or to header information. The image data may include information that identifies what the frame number is and which session of dynamic imaging the frame belongs to. This allows displaying a first image or segmented display in units of intermittent imaging.

In addition to the stop operations illustrated in FIG. 12 to FIG. 14, the console 10 may automatically determine that the imaging ends when any of the following conditions (1) to (6) is satisfied.
(1) The total irradiation dose reaches a predetermined irradiation dose.
(2) The total of the imaging period and the imaging suspension period has reached a predetermined period.

For example, an upper limit is set to three minutes, and imaging and pausing can be performed for any number of times during three minutes.
(3) The suspension time of intermittent imaging has reached a predetermined time.

Thus, when the suspension period is equal to or longer than the predetermined period, the imaging can be ended in view of imaging quality, and the reset processing can be performed.
(5) The number of times of intermittent imaging has reached a predetermined number.

A technician may not intuitively recognize the period and the dose. By managing intermittent imaging based on the number of times the intermittent imaging can be performed, the technician can easily recognize the timing to end the imaging.
(5) The number of frames in one time of dynamic imaging has exceeded a specified value.
(6) The user makes an ending operation, such as pressing a switch of the imaging device 2 or manipulating the screen of the console 10.

The setting on the above (1) to (5) may be changed for each imaging order or each frame rate by manipulating the operation part 32, for example.

The imaging order in the normal dynamic imaging mode and the imaging order in the intermittent imaging mode of the present embodiment may be changed in any of the following (1) to (3).
(1) The setting range of irradiation conditions including a tube voltage (kV), a tube current (mA), and a mAs value of X-rays

In dynamic imaging in the intermittent imaging mode, deterioration of the imaging device 2 can be suppressed by narrowing the settable range in order to maintain the “Ready” state of the imaging device 2 for a long time.
(2) Changing warm-up time of the imaging device 2

In the intermittent imaging mode including a long imaging time, offset correction can be performed by one time offset calibration.
(3) Changing the remaining battery capacity that allows wireless imaging

In the intermittent imaging mode including a long imaging time, running out of a battery in the middle of imaging can be prevented.

When either of the following conditions (1) and (2) is satisfied before the start of the intermittent imaging mode, the console 10 may display a warning or an error on the display part 30 or may prohibit the start of the intermittent imaging mode.
(1) The relation “Imaging period>Synchronization maintenance time” is satisfied.

The imaging period is a predetermined imaging time in the intermittent imaging mode including still image capturing and dynamic imaging. The synchronization maintenance time is a time during which the imaging device 2 can be accurately synchronized with the parent medical cart 1 when the imaging device 2 is removed from the medical cart 1 and wirelessly connected to the medical cart 1.
(2) The relation “the number of capturable frames>the number of times the radiation source 22 can emit radiation” is satisfied.

Although the preferred embodiments of the present disclosure have been described in detail with reference to the accompanying drawings, the technical scope of the present disclosure is not limited to such examples. Further, those to which various modification examples and improvements have been applied naturally belong to the technical scope of the present disclosure within the category of the technical idea described in the scope of the claims of those skilled in the art.

1. A dynamic imaging system comprising:
a radiation source;
a radiation detector; and
a hardware processor, wherein:
the dynamic imaging system obtains a still image consisting of a single frame and a dynamic image consisting of multiple frames by irradiating a subject with radiation emitted by the radiation source and detecting the radiation transmitted through the subject by the radiation detector.
the hardware processor controls the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period, and
the hardware processor ends the one session of imaging, based on a total number of frames of the still image and the dynamic image.

2. The dynamic imaging system according to claim 1, wherein the hardware processor ends the imaging when the total number of frames reaches a first specified number of frames.

3. The dynamic imaging system according to claim 1, wherein the hardware processor ends the imaging when the total number of frames reaches a second specified number of frames in the imaging suspension period.

4. The dynamic imaging system according to claim 3, wherein the hardware processor is capable of changing the second specified number of frames for each session of imaging from one time of imaging start to one time of imaging end.

5. The dynamic imaging system according to claim 1, wherein the hardware processor ends the imaging when a specified imaging period has elapsed since the one time of imaging start.

6. The dynamic imaging system according to claim 1, wherein the radiation detector performs an image reading operation at an identical frame rate in obtaining the still image and the dynamic image.

7. The dynamic imaging system according to claim 1, wherein the dynamic imaging system can obtain the still image and/or the dynamic image multiple times during the one session of imaging from the one time of imaging start to the one time of imaging end.

8. The dynamic imaging system according to claim 1, wherein:
the hardware processor sets an imaging condition for the radiation source, and
the hardware processor does not accept a change in the imaging condition between the one time of imaging start to the one time of imaging end.

9. The dynamic imaging system according to claim 8, wherein the hardware processor sets the identical imaging condition for obtaining the still image and the dynamic image.

10. The dynamic imaging system according to claim 1, further comprising a notification unit that notifies information on the total number of frames.

11. A radiographic imaging method for a dynamic imaging system that includes a radiation source, a radiation detector, and a hardware processor and that obtains a still image consisting of a single frame and a dynamic image consisting of multiple frames by irradiating a subject with radiation emitted by the radiation source and detecting the radiation transmitted through the subject by the radiation detector, the method comprising:
controlling the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period, and
ending the one session of imaging, based on a total number of frames of the still image and the dynamic image.

12. A non-transitory computer-readable storage medium storing a program for a dynamic imaging system that includes a radiation source, a radiation detector, and a computer and that obtains a still image consisting of a single frame and a dynamic image consisting of multiple frames by irradiating a subject with radiation emitted by the radiation source and detecting the radiation transmitted through the subject by the radiation detector, wherein the program causes the computer to:
control the radiation source and the radiation detector to operate in an intermittent imaging mode in which the still image and/or the dynamic image are obtained multiple times during one session of imaging from one time of imaging start to one time of imaging end, based on a predetermined imaging period and a predetermined imaging suspension period and
end the one session of imaging, based on a total number of frames of the still image and the dynamic image.
